(19) United States
(12) Patent Application Publication
(10) Pub. No.: US 2006/0069312 A1
(43) Pub. Date: Mar. 30, 2006

(54) System for retaining optical clarity in a medical imaging system

(75) Inventor: John P. O'Connor, Andover, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SCIMED Life Systems, Inc., Maple Grove, MN (US)

(21) Appl. No.: 10/955,920

(22) Filed: Sep. 30, 2004

(51) Int. Cl. A61B 1/06

(52) U.S. Cl. 600/176

(57) ABSTRACT

In one aspect, the present invention is a medical imaging device that retains optical clarity when used in a patient. The medical imaging device includes a shaft having a distal end and a proximal end and an imaging assembly disposed at the distal end of the shaft. The imaging assembly includes at least one imaging lens and at least one illumination port lens. The outer surface of at least one lens in the imaging assembly is coated with a biocompatible composition comprising a hydrophilic material that retains the optical clarity of the medical imaging device. In another aspect, the invention provides a kit for retaining the optical clarity of a medical imaging system.

### FIELD OF THE INVENTION

The present invention generally relates to medical devices and in particular to a system for retaining optical clarity in a medical imaging system.

### BACKGROUND OF THE INVENTION

As an aid to the early detection of disease, it has become well established that there are major public health benefits from regular endoscopic examinations of a patient's internal structures such as the alimentary canals and airways, e.g., the esophagus, lungs, colon, uterus, and other organ systems. Conventional imaging endoscopes used for such procedures generally include an illuminating mechanism such as a fiber optic light guide connected to a proximal source of light or light-emitting diodes (LEDs) at the distal tip of the endoscope, and an imaging means such as an imaging light guide to carry an image to a remote camera or eye piece or a miniature video camera within the endoscope itself to produce an image that is displayed to the examiner. In addition to examinations, imaging endoscopes are also commonly used to perform surgical, therapeutic, diagnostic or other medical procedures under direct visualization. Most endoscopes include one or more working channels through which medical devices such as biopsy forceps, snares, fulguration probes and other tools may be passed in order to perform a procedure at a desired location in the patient's body.

During the use of an imaging endoscope, blood, tissue, fecal material, surgical debris or other matter can lodge on the imaging lens, thereby obscuring the field of view and preventing the ability of an examiner to view a clear image of the patient's tissue. This is especially common when the endoscope is used for colonoscopy in a poorly prepared patient. During such a procedure the endoscope must be removed from the patient to clean the debris off the lens, causing an interruption in the procedure and potential discomfort to the patient. Because of the inconvenience of removing and cleaning an endoscope during a procedure, most endoscopes are equipped with a lens wash apparatus with a flushing cap that sprays a stream of fluid over the observation lens to flush away the debris. However, there are several disadvantages to a lens wash apparatus. Such an apparatus adds complexity to the endoscope device, increases the outer diameter of the device at the tip, and is subject to malfunction. Also, the lens wash apparatus may not successfully remove debris that has become adhered to the lens. Therefore, there is a need for a device and method that retains the optical clarity of an endoscope lens during a clinical procedure without necessarily requiring a lens wash apparatus or the removal of the endoscope from a patient's body during the procedure.

### SUMMARY OF THE INVENTION

To address these and other problems, the present invention is a system for retaining the optical clarity of a medical imaging device. The medical imaging device includes a shaft having a distal end and a proximal end and an imaging assembly disposed at the distal end of the shaft. The imaging assembly includes at least one imaging lens and at least one illumination port lens. The outer surface of at least one lens in the imaging assembly is coated with a biocompatible composition comprising a hydrophilic material that retains the optical clarity of the medical imaging device.

In another aspect, the invention is a kit for retaining the optical clarity of a medical imaging system. The kit includes a medical imaging device with a shaft having a distal end and a proximal end and an imaging assembly disposed at the distal end of the shaft. The imaging assembly includes at least one imaging lens and at least one illumination port lens. The kit also includes a biocompatible composition comprising a hydrophilic material packaged in a sterile container and written instructions describing a method of applying a coating of the biocompatible composition to the outer surface of at least one lens in the imaging assembly just prior to clinical use of the medical imaging device.

In another aspect, the invention is a method for retaining the optical clarity of an imaging endoscope system. The method includes coating at least one lens of an imaging endoscope with a biocompatible composition comprising a hydrophilic material and packaging the imaging endoscope into a removable sterile wrapper.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram illustrating a medical imaging device that retains the optical clarity of a medical imaging system in accordance with an embodiment of the present invention;

FIG. 2A illustrates a distal end of a medical imaging device that includes an imaging assembly that retains the optical clarity of a medical imaging system in accordance with an embodiment of the present invention;

FIG. 2B illustrates an imaging assembly having an imaging lens and multiple illumination port lenses in accordance with an embodiment of the present invention;

FIG. 3A shows a perspective view of a representative imaging lens assembly having an imaging lens with a coating for retaining optical clarity in accordance with an embodiment of the present invention;

FIG. 3B shows a perspective view of a representative imaging lens having a coating for retaining optical clarity in accordance with an embodiment of the present invention; and

FIG. 3C is a cross-section of the coated imaging lens shown in FIG. 3B.

### DETAILED DESCRIPTION OF THE PREFERRED EMBODIMENT

Generally described, the present invention is a system for improving the optical clarity of a medical imaging device. Although the present invention is useful with a variety of medical imaging devices, it is particularly useful with an endoscope of the type that includes a shaft with a distal and proximal end and an imaging assembly disposed at the distal end of the shaft. The imaging assembly includes at least one imaging lens and at least one illumination port lens. The outer surface of at least one lens in the imaging assembly is coated with a biocompatible composition having a hydrophilic material that retains the optical clarity of the medical imaging device. The lens coating retains the optical clarity of the device by preventing contaminants such as bodily fluids, fecal material, surgical debris and the like from sticking to the lens. In some embodiments, the coating is applied at the time of manufacture. In other embodiments, the medical imaging device further comprises a removable sterile wrapper that covers the coated lens. In other embodiments, the composition comprising a hydrophilic material is provided in a kit with the device and a coating of the composition is applied to a lens on the device just prior to introducing the device into a living body.

The medical imaging devices, kits and methods of the present invention may be used for any medical procedure that involves inserting a medical imaging device into a living body. Illustrative examples of medical imaging devices include imaging endoscopes (such as a colonoscope, gastroscope, laparoscope or arthroscope), imaging catheters, fiber optic guide wires and the like. In some embodiments, the medical imaging devices of the invention are reusable while in other embodiments, the devices are disposable single use devices such as single use imaging endoscopes.

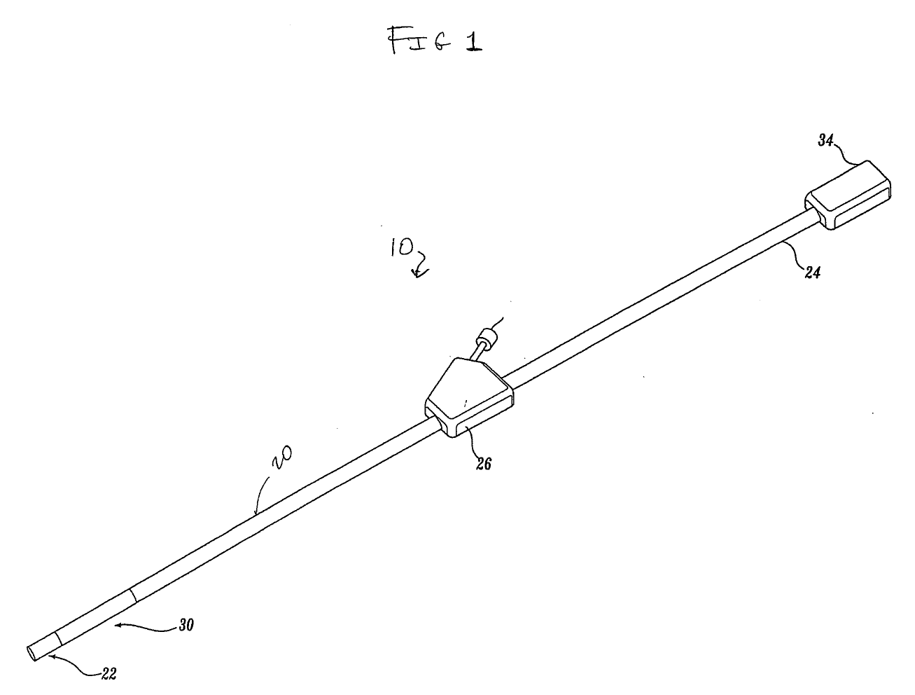

FIG. 1 illustrates one embodiment of a representative medical imaging device 10 of the present invention. The medical imaging device 10, shown here as an imaging endoscope, comprises a shaft 20 that contains one or more lumens for the purpose of performing endoscopic procedures and facilitating the insertion and extraction of fluids, gases and/or surgical or diagnostic devices into and out of a living body. The shaft 20 comprises a distal end 22 that is advanced into a patient's body and a proximal end 24 that may be connected via a proximal connector 34 to a control cabinet (not shown). Proximal to the distal end 22 is an articulation joint 30 that provides sufficient flexibility to the distal section of the shaft such that the distal end 22 can be directed to bend in any direction desired about the circumference of the distal end 22. In the embodiment shown, the imaging medical device 10 also includes a breakout box 26 that is positioned approximately midway along the length of the endoscope shaft 20. The breakout box 26 provides a mechanism to allow a physician to grasp the shaft 20 as well as provides an entrance to a working lumen within the shaft. Although the imaging medical device illustrated in FIG. 1 is an endoscope, it will be understood by those of ordinary skill in the art that the medical imaging device of the present invention may be any medical imaging device, not limited to endoscopes.

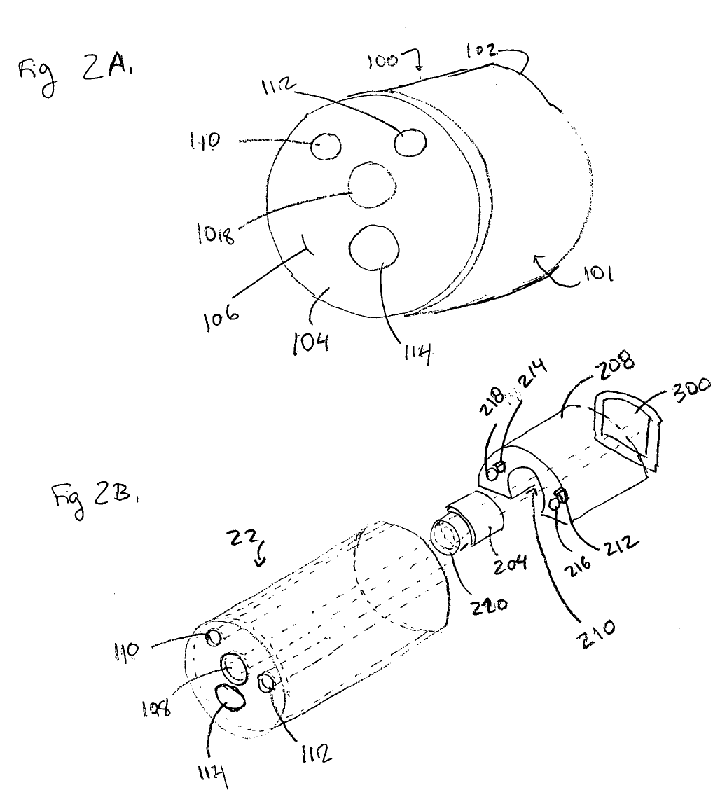

FIG. 2A shows further detail of one embodiment of the distal end 22 of the medical imaging medical device 10. As shown in FIG. 2A, the distal end 22 includes a cylindrical imaging assembly 100 that comprises a cylinder 101 having a proximal end 102 and a distal end 104. A distal face 106 of the distal end 104 of the cylinder 101 includes a number of ports, including an imaging lens port 108 containing an imaging lens, two or more illumination ports 110 and 112 containing illumination port lenses, and an access port 114 that defines the entrance to the working channel lumen.

FIG. 2B shows further detail of the imaging assembly at the distal end 22 of the medical imaging device 10. As shown in FIG. 2B, fitted within the imaging lens port 108 is an imaging lens 220 that is part of a cylindrical lens assembly 204. The lens assembly 204 is fitted within a heat exchanger 208. An image sensor 300 is secured to the proximal end of the heat exchanger 208 to record images focused by the lens assembly 204. The image sensor 300 is preferably a CMOS imaging sensor or other solid state imaging device such as a charge-coupled device (CCD).

The heat exchanger 208 comprises a semi-circular section having a concave recess 210 into which the cylindrical imaging lens assembly 204 can be fitted. The concave recess 210 holds the position of the imaging lens assembly 204 in directions perpendicular to the longitudinal axis of endoscope, thereby only permitting the imaging lens assembly 204 to move along the longitudinal axis of the endoscope. Once the imaging lens assembly 204 is positioned such that it is focused on the image sensor 300, the imaging lens assembly 204 is fixed in the heat exchanger 208 with an adhesive.

With continued reference to FIG. 2B, a pair of light emitting diodes (“LEDs”) 212 and 214 are bonded to a circuit board (not shown) that is bonded to a front surface of the heat exchanger 208 such that a channel is formed behind the circuit board for the passage of a fluid or gas to cool the LEDs. With the imaging lens assembly 204, the LEDs 212, 214, the image sensor 300, and associated control circuitry (not shown) secured in the heat exchanger 208 can be fitted within the cylinder 101 to complete the imaging assembly as shown in FIG. 2B.

The LEDs 212 and 214 are fitted behind the illumination ports 110 and 112 as shown in FIG. 2B. Each illumination port 110, 112 additionally contains an illumination window or lens 216, 218 covering the LEDs 212 and 214. The LEDs 212 and 214 may be high intensity white light sources or may comprise colored light sources such as infrared (IR) red, green, blue or ultra-violet (UV) LEDs. Although the embodiment of the distal end 22 shown in FIG. 2B shows two LEDs 212 and 214 that are positioned on either side of the lens assembly 204, it will be appreciated that additional LEDs could be used and corresponding changes made to the shape of the illumination ports 110 and 112, positioned in front of the LEDs. As an alternative to LEDs, the light source for the medical imaging device 10 may be external to the endoscope such that the illumination light is delivered to the illumination port with a fiber optic bundle of a light carrying device.

In operation, the medical imaging device 10 captures images of patient tissue that are illuminated by the LED light source(s). Debris and bodily fluids cover the LED illumination ports and may interfere with the intensity of the illumination emitted from the illumination ports 110 and 112. Therefore, in accordance with some embodiments of the invention, the one or more illumination port lenses 216, 218 are coated with a biocompatible composition comprising a hydrophilic material to retain the optical clarity of the medical imaging device 10.

Because the optical components of the imaging assembly are coated to retain their optical clarity, in some embodiments, the imaging assembly does not require a lens wash apparatus, although one may be optionally provided. Therefore, the medical imaging device 10 has many advantages over a medical imaging device that require a lens wash apparatus. For example, the medical imaging device 10 may be constructed with a distal end 22 having a smaller diameter than a medical imaging device that requires a lens wash apparatus. Moreover, the simplified distal end 22 reduces manufacturing costs due to the elimination of a lens wash apparatus and associated fluidics inside a medical imaging device that are associated with a lens wash apparatus.

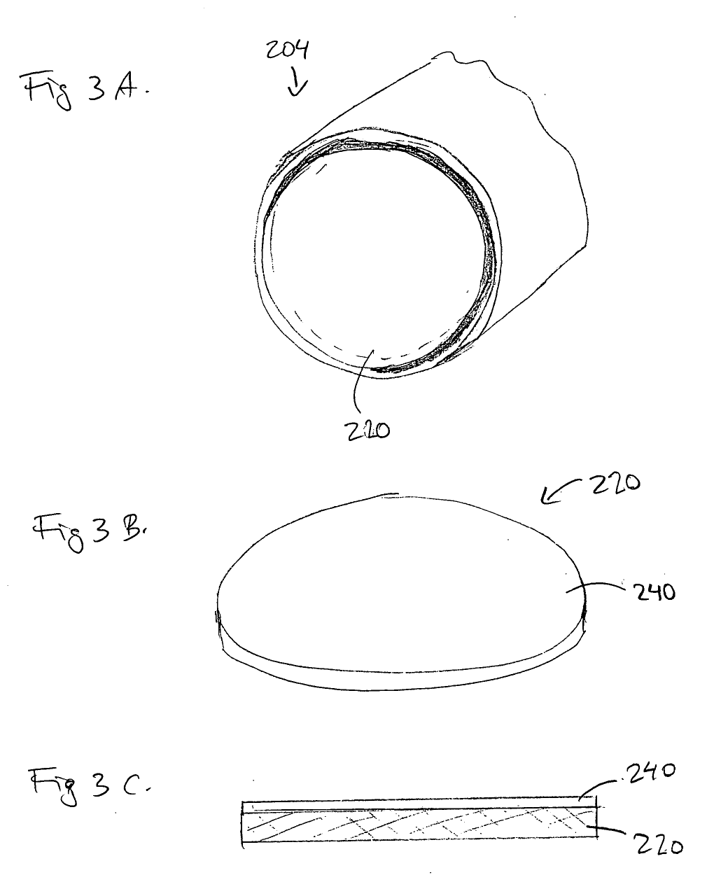

FIG. 3A shows one embodiment of the imaging lens assembly 204 with the imaging lens 220. The imaging lens 220 and the illumination port lenses 216, 218 may be made of any suitable material such as glass or clear plastic. Preferably, the imaging lens 220 provides a 140° field of view with f-theta distortion and a f/8 aperture. In accordance with the FDA Guidance Document for Endoscopes, the resolution of the imaging lens 220 should be 5 line pairs per millimeter or better for an object 10 mm distant from the distal end 22.

FIG. 3B shows a perspective view of the imaging lens 220 having a biocompatible coating 240 comprising hydrophilic material coated on the outer surface of the lens 220 in accordance with an embodiment of the invention. FIG. 3C is a cross sectional view of the lens 220 shown in FIG. 3B, that illustrates the coating 240 on the outer surface of the imaging lens 220. The biocompatible coating 240 is formed on the outer surface of the imaging lens 220 with a composition comprising any suitable hydrophilic material that maintains the optical clarity of the medical imaging device 10. The hydrophilic properties of the biocompatible composition in the coating 240 create a sheeting effect when bodily fluids and/or debris contact the coated outer surface of the lens 220, allowing contaminants to be carried away from the lens surface.

In some embodiments, the hydrophilic material in the lens coating 240, such as a hydrophilic polymer or hydrogel, forms a lubricious coating when contacted with a liquid. A hydrophilic polymer useful in the biocompatible composition may comprise monomer units from one or more monomers having organic acid functional groups, such as, for example, acrylic acid, methacrylic acid and isocrontonic acid. In addition, the hydrophilic polymer may contain monomer units from at least one hydrophilic monomer without any organic acid functional groups, such as vinylpyrrolidone and acrylamide. Exemplary biocompatible hydrophilic polymers include, but are not limited to, poly(N)-vinyl lactams, such as polyvinylpyrrolidone (PVP) and the like, polyethylene oxide (PEO), polypropylene oxide (PPO), polyacrylamides, cellulosics, such as methyl cellulose and the like, polyacrylic acids, such as acrylic and methacrylic acids and the like, polyvinyl alcohols, and polyvinyl ethers and the like. For example, the hydrophilic polymer may be acrylic acid-acrylamide copolymer (supplied by Allied Colloids as Versicol WN33). In some embodiments, the biocompatible composition comprises the HYDROPASS™ hydrophilic coating available from Boston Scientific Corporation, of Natick Mass., and described in U.S. Pat. Nos. 5,702,754, and 6,048,620, which are herein incorporated by reference.

In some embodiments, the biocompatible composition forms a lens coating that is covalently attached to the lens. The composition can be covalently attached using any suitable method, such as the method described in U.S. Pat. No. 5,702,754 for covalently attaching the HYDROPASS™ hydrophilic composition to a substrate. Briefly described, an imaging lens 220 or an illumination port lens 216, 218 is coated with a first aqueous coating composition comprising an aqueous dispersion or emulsion of a polymer having organic acid functional groups and a polyfunctional crosslinking agent having functional groups being capable of reacting with organic acid groups. The coating is dried on the lens to obtain a substantially water-insoluble coating layer still including functional groups being reactive with organic acid groups. The dried, coated lens is then contacted with a second aqueous coating composition comprising an aqueous solution or dispersion of a hydrophilic polymer having organic acid functional groups. The combined coating is then dried with the hydrophilic polymer thereby becoming bonded to the polymer of the first coating composition through the crosslinking agent. Typically, the polymer in the first coating composition is selected from homo- and copolymers including polyurethanes, polyacrylates, polymethacrylates, polyisocrotonates, epoxy resins, acrylate-urethane copolymers and combinations thereof having organic acid functional groups. Examples of useful polyfunctional crosslinking agents having functional groups being capable of reacting with organic acid groups include polyfunctional aziridines and polyfunctional carbodimides. Hydrophilic polymers useful in the second coating are the same as previously described above. This process provides a covalently bonded lubricous, hydrophilic coating with excellent wear resistance. The coating can be applied in thin layers so as not to affect the optical clarity of the observation lens. The coating may be applied on a lens with a coating thickness suitable to retain optical clarity of the lens. A non-limiting example of a suitable coating thickness is from about 100 microns to less than 1 micron, and more preferably from about 10 microns to less than 1 micron.

Alternatively, self-cleaning compositions comprising hydrophilic materials may be applied to a lens to create a non-covalently bound coating. Such non-covalently bound coatings may be preferable for single use imaging devices.

The biocompatible composition comprising a hydrophilic material may be applied to the lens to form a coating at the time of manufacture of the medical imaging device. The coating may be applied to the lens either before or after the assembly of the imaging device by spraying, swabbing, brushing or dipping the outer surface of the lens with the composition comprising a hydrophilic material. In such embodiments, after the application of the composition, the device is sterilized and may be packaged with a removable sterile wrapper.

In another aspect, the present invention provides a kit for retaining optical clarity in a medical imaging system. The kit includes a medical imaging device with an imaging assembly having at least one imaging lens and at least one illumination port lens and a biocompatible composition comprising a hydrophilic material packaged in a sterile container. The kit may also include written indicia describing a method of applying a coating of the biocompatible composition to the outer surface of at least one lens in the imaging assembly just prior to clinical use of the medical imaging device. The devices and biocompatible compositions comprising hydrophilic material disclosed herein are useful in the kit of the present invention.

The biocompatible composition comprising a hydrophilic material is preferably packaged in a sterile container in a microbiologically stable form. Microbiological stability can be achieved by any suitable means, such as by freezing, refrigeration, or lyophilization of the composition. Sterilization of the composition can be achieved by any suitable means such as by heat, chemical or filtration mediated sterilization, and/or by the addition of antimicrobial agents.

In another aspect, the present invention provides methods for retaining the optical clarity of an imaging endoscope system. The medical imaging devices described herein are useful in the methods of this aspect of the invention. The method comprises coating at least one lens of an imaging endoscope with a biocompatible composition comprising a hydrophilic material and packaging the imaging endoscope with at least one coated lens into a removable sterile wrapper. The coated lens retains optical clarity of the imaging system. In some embodiments, the lens is coated with the biocompatible composition at the time of manufacture. In some embodiments the method further comprises packaging the assembled imaging device with at least one coated lens into a removable sterile wrapper prior to shipment.

While the preferred embodiment of the invention has been illustrated and described, it will be appreciated that various changes can be made therein without departing from the scope of the invention.

The embodiments of the invention in which an exclusive property or privilege is claimed are defined as follows:

1. A medical imaging device comprising:
a shaft having a distal end and a proximal end and an imaging assembly disposed at the distal end of the shaft,
wherein the imaging assembly comprises at least one imaging lens and at least one illumination port lens and wherein the outer surface of at least one lens in the imaging assembly is coated with a biocompatible composition comprising a hydrophilic material.

2. The medical imaging device of claim 1, wherein the distal end of the shaft does not include a lens wash apparatus.

3. The medical imaging device of claim 1, wherein the hydrophilic material becomes lubricous when contacted with an aqueous medium.

4. The medical imaging device of claim 1, wherein the hydrophilic material is an acrylic acid-acrylamide copolymer.

5. The medical imaging device of claim 1, wherein the biocompatible composition comprising hydrophilic material is covalently bound to at least one lens.

6. The medical imaging device of claim 1, wherein the medical imaging device is a single use endoscope.

7. The medical imaging device of claim 1, wherein the medical imaging device is a reusable endoscope.

8. The medical imaging device of claim 1, wherein the coated lens is an imaging lens.

9. The medical imaging device of claim 1, wherein the coated lens is an illumination port lens.

10. An imaging assembly for use in a medical imaging device, the imaging assembly comprising:
at least one imaging lens and at least one illumination port lens, wherein the outer surface of at least one lens in the imaging assembly is coated with a biocompatible composition comprising a hydrophilic material.

11. A kit for retaining the optical clarity of a medical imaging system, said kit comprising:
i) a medical imaging device comprising a shaft having a distal end and a proximal end and an imaging assembly disposed at the distal end of the shaft, wherein the imaging assembly comprises at least one imaging lens and at least one illumination lens;
ii) a biocompatible composition comprising a hydrophilic material packaged in a sterile container; and
iii) written indicia describing a method of applying a coating of the biocompatible composition to the outer surface of at least one lens in the imaging assembly just prior to clinical use of the medical imaging device.

12. The kit of claim 11, wherein the distal end of the shaft does not contain a lens wash apparatus.

13. The kit of claim 11, wherein the hydrophilic material becomes lubricious when contacted with an aqueous medium.

14. The kit of claim 11, wherein the hydrophilic material is an acrylic-acrylamide copolymer.

15. The kit of claim 11, wherein the biocompatible composition comprising hydrophilic material is capable of covalently binding to at least one lens.

16. The kit of claim 11, wherein the medical imaging device is a single use endoscope.

17. The kit of claim 11, wherein the medical imaging device is a reusable endoscope.

18. A method for retaining the optical clarity of an imaging endoscope system, said method comprising:
coating at least one lens of an imaging endoscope with a biocompatible composition comprising a hydrophilic material; and
packaging the imaging endoscope with at least one coated lens into a removable sterile wrapper.

19. The method of claim 18, wherein the imaging endoscope does not include a lens wash apparatus.

20. The method of claim 18, wherein the hydrophilic material becomes lubricious when contacted with an aqueous medium.

21. The method claim 18, wherein the hydrophilic material is an acrylic-acrylamide copolymer.

22. The method of claim 18, wherein the coating is applied to at least one imaging lens.

23. The method of claim 18, wherein the coating is applied to at least one illumination lens.

24. The method of claim 18, wherein the imaging endoscope is a single use endoscope.

25. The method of claim 18, wherein the imaging endoscope is a reusable endoscope.
